(19) United States
(12) Patent Application Publication
(10) Pub. No.: US 2025/0292914 A1
(43) Pub. Date: Sep. 18, 2025

(54) SYSTEM AND METHOD FOR AI ASSISTED THERAPEUTIC MICRO-INTERVENTIONS IN CARE AT HOME

(71) Applicant: Lifeguard Health Networks, Inc., Radnor, PA (US)

(72) Inventors: Martin Carty, Wayne, PA (US); Stephen Malaby, Devon, PA (US)

(21) Appl. No.: 19/068,861

(22) Filed: Mar. 3, 2025

Related U.S. Application Data
(60) Provisional application No. 63/565,659, filed on Mar. 15, 2024.
(60) Provisional application No. 63/565,662, filed on Mar. 15, 2024.

(51) Int. Cl. G16H 50/70; G16H 10/60

(52) U.S. Cl. CPC G16H 50/70; G16H 10/60

(57) ABSTRACT

A distributed healthcare network system stores intervention, context, and communication rules in databases, defining responses to patient-related events. Context includes factual circumstances tied to the patient. The system constructs a distributed ledger for each patient, incorporating their profile, therapeutic events, and context indicators based on predefined rules. Interventions are selected according to patient context and communicated to relevant network nodes, with records added to the ledger. AI models trained on patient data help identify subtle scenarios from context indicators and suggest alternative actions. Intervention rules rely on fixed thresholds and AI-driven analysis to determine appropriate responses. The ledger, distributed across the network, ensures transparency, enhances communication among patients, providers, and payers, and streamlines healthcare transactions. This method enables efficient decision-making, ensures accurate documentation, and improves healthcare coordination by maintaining an immutable, accessible record of patient interactions, interventions, and contextual factors.

### CROSS-REFERENCE TO RELATED APPLICATIONS

This application claims the benefit of U.S. Provisional Appl. No. U.S. 63/565,659 filed Mar. 15, 2024 and U.S. Provisional Appl. No. 63/565,662 filed Mar. 15, 2024, both of which are incorporated herein by reference in their entireties. This application is filed concurrently with U.S. Appl. No. ______ entitled “System and Method for Therapeutic Event Management and Value Attribution (Proof of Value) in Decentralized Health Information Network” and U.S. application Ser. No. 19/068,782 and entitled “System and Method for Protecting Patients' Therapeutic Treatment Context Used for Training Artificial Intelligence Systems” (Atty. Dkt. No. 266-0004US), which is also incorporated herein by reference in their entireties.

### TECHNICAL FIELD

This disclosure relates generally to therapeutic monitoring and treatment of medical patients primarily outside of a hospital setting.

### BACKGROUND OF THE DISCLOSURE

Research studies and anecdotal reports agree that at-home medical treatment and recovery may be safer, cheaper, and more effective than traditional hospital care, but only if the patients are closely monitored. The benefits of home care may be especially important for patients who are vulnerable to infections that are common in hospitals or to other complications of inpatient care. In addition, some studies indicate that well-monitored healthcare at home may be more effective at improving patient outcomes than traditional hospitalization. Furthermore, most experts agree that home healthcare can save money for both patients and payer organizations, including governments.

Healthcare outside of a hospital or other dedicated medical environment benefits from fulsome patient surveillance because surveillance allows healthcare professionals to more granularly evaluate patient progress as well as a patient's adherence and response to therapies. Not surprisingly, there is a medical surveillance marketplace offering labor, techniques, and technology to monitor patients' health and safety when those patients are away from medical facilities. The technology employed in marketable solutions may include hardware, such as sensors, cameras, and other devices, which can track a patient's vitals, wellness, movements, or other activities. There is also software at either or both of the system or client application level, which can help a patient report on subjective matters and help enforce the proper administration of therapies. In all, these technologies provide healthcare professionals with real-time or near-real-time information about a patient's condition and other medically relevant activities, which allows more accurate and timely medical interventions.

Certain solutions for remote patient monitoring are static, hard-coded data models that require universal updates across all parties and global adherence to a single definition. This monolithic approach limits best practice logic and/or customization that may be added by users (e.g., providers or other participants in the healthcare marketplace) with more relevant knowledge regarding the needs of patient cohorts or individual patients. By hindering the flexibility of downstream healthcare providers, researchers, and administrators, such static and hard-coded models lock all participants into the same data model universally. That result may hinder differentiation of remote surveillance services, may limit the availability of more customized patient-centric care, and may potentially burden the clinical teams with manual application of customizations.

Currently, healthcare services are shifting to value-based contracts between insurers, health plans, and managed care teams. These entities want to address the inherent challenges in coordinating care for at-risk Medicare populations outside of clinical settings. Accordingly, providers and payers seek to improve performance and meet quality and cost targets by getting ahead of clinical risks, engaging patients, and effectively optimizing healthcare costs and revenue cycle. Unfortunately, the present state of the art limits the information available to payers and providers for satisfying value-based contracts. In fact, good evidence of care occurring at a patient's home (tolerance, adherence, and risk) is not available to providers for them to satisfy value-based contracts. Typically, payers and providers only have access to the clinical observations captured episodically in the Electronic Medical Record (EMR).

Currently, adjustments to a patient's care plan occur primarily in clinic appointments. As a result, the efficacy of at-home care is assessed infrequently and is often inaccurately assessed when recalled later. At worse, a medical emergency may be prevented if the at-home care reveals symptoms that are leading indicators of mounting risk.

### BRIEF DESCRIPTION OF DRAWINGS

FIG. 1 illustrates an example of a health information network according to the present disclosure.

FIG. 2 illustrates a proof of value system utilized in a distributed health information network system.

FIG. 3 illustrates a flowchart of a proof of value process according to the present disclosure.

FIG. 4A illustrates an example of the distributed network system being used for therapeutic tolerance and safety.

FIG. 4B illustrates a portion of the proof of value process for the therapeutic tolerance and safety example in FIG. 4A.

FIG. 5A illustrates an example of the distributed network system being used for therapeutic micro-intervention.

FIG. 5B illustrates a portion of the proof of value process for the therapeutic micro-intervention example in FIG. 5A.

FIG. 6 illustrates a natural language processing platform for creating training data to be used in training an AI model in a computing environment.

FIG. 7 illustrates a process for training and deploying a deep neural network.

FIG. 8 schematically illustrates components of network nodes for the distributed network system.

### DETAILED DESCRIPTION OF THE DISCLOSURE

U.S. Publication No. US2014/0207486 to Carty et al. and assigned to Lifeguard Health Networks, Inc. is hereby incorporated by reference in its entirety as background information and to aid in the understanding of more advanced concepts discussed herein.

Systems and methods described herein disclose a network enabled (e.g., TCIP/IP enabled) continuity of care network, including one or more computing devices, sensors, and systems for communications and data sharing. These networks enable people on a caregiver team to receive and send medically relevant communications with a patient or a patient representative in response to health events, incidents, questions, observations, trends, and the like. A continuity of care network may be doctor-initiated and may be payer or insurer approved, and the continuity of care network is part of one possible network arrangement of this disclosure. In many arrangements, patients are invited onto the system by a healthcare provider or a medical-related entity with a relationship to the patient's care. Because healthcare often involves a support team around the patient, arrangements of the disclosed network system allow patients to invite family, friends, and other health advocates to join the system as a patient representative. One or more computing devices for each system participant may be joined into the network/system to run or access software related to the techniques herein and access associated functionality.

The disclosed system proposes technology and solutions through a collaborative care network, including devices having software made available for use by various participants in the healthcare ecosystem and marketplace. For example, depending upon the arrangement, software delivering all or part of the solution may reside on mobile devices, computers, thin clients, or intelligent devices of any kind that are commonly used by patients, patient representatives, healthcare professionals, insurers, government, medically related entities such as institutions, health systems, research entities, and all manner of healthcare provider organizations. As implemented, the software may have multiple widespread application areas. Healthcare providers and other healthcare related entities (such as insurers and institutes) may use a version of software (e.g., application) adapted for endpoint operating systems such as Microsoft Windows or Mac OS, or server operating systems (e.g., for SaaS solutions) such as Linux or Windows Server. In addition, some embodiments allow providers and other entities access to software functionality through (i) mobile applications (e.g., IOS, Android, Java); (ii) a web portal accessed through a web browser on any device; or (iii) any information access and/or delivery system such as virtual or augmented reality tools. In some embodiments, these options may be facilitated through known client-server protocols using computer servers in a data center, public cloud, or other known computing resources.

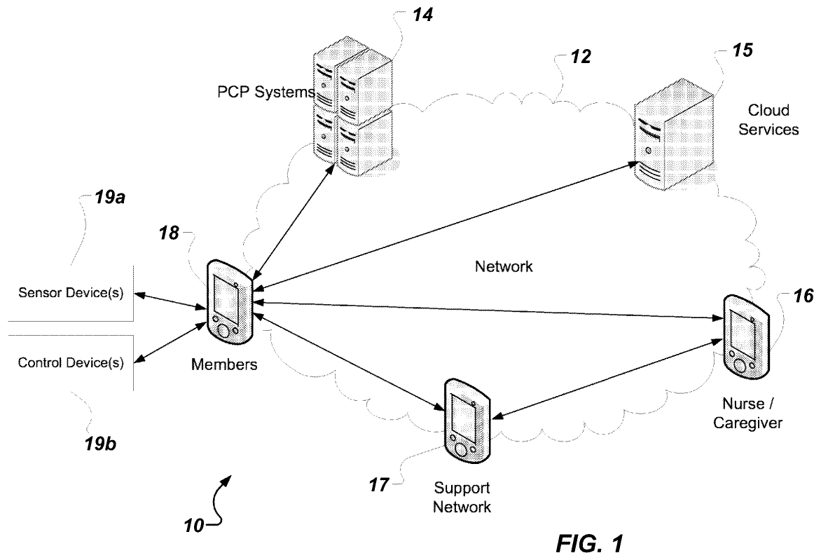

FIG. 1 shows an exemplary continuity of care network system 10. As shown in FIG. 1, the network system 10 is decentralized with computing power, data capture and monitoring and other functionality distributed and available at each of the various components. Patients or their representatives typically interface with/connect to the network system 10 with patient devices 18. Patient device 18 may be an edge-of-network computing device (e.g., a computer or mobile device such as a smartphone or tablet), optionally configured to monitor patient data based upon a specified health service policy. For example, a device may monitor the patient's biotelemetry, location, or other items depending upon the capability of the device. The patient's device (e.g., client device) 18 may also receive inputs from other devices such as sensors 19a or control devices 19b. Patient device 18 may transmit patient data across one or more networks 12 to other devices and systems in the network system 10, such as one or more representative devices (e.g., client device) 17, a nurse or other caregiver device (e.g., client device) 16, one or more primary care provider systems 14, and servers/databases such as cloud services systems 15. Of course, network system 10 may include any number of additional devices and systems or may omit one or more devices or systems shown in FIG. 1.

Patient representative devices (e.g., client device) 17 may be used by those appointed by or on behalf of the patient, such as trusted family, friends, and other caregivers of a patient to assist with daily therapy compliance, program adherence, communications, health management, and the like. Network system 10 may include multiple patient representative devices 17 coupled to (i.e., in communication with) a patient device 18, thereby providing a few-to-one or many-to-one support network for the patient. By including multiple people's devices with customized privacy and intervention alert levels within the network, the responsibility of monitoring patient data may be distributed amongst a patient's family or other care representatives or network participants. Additionally, such a group may provide monitoring redundancy, for example if one representative does not have access (e.g., internet access) others in the patient's support network may monitor patient's data. In addition, aspects of the network system 10 associated with a given patient may be dynamic, expanding and contracting in response to a patient's needs, schedule, and the like.

Representative devices 17 and nurse/caregiver device 16 may be any type of computing devices, such as any computing endpoint or mobile computing device, thereby enabling monitoring the patient substantially all times. Devices on the network system 10 may receive substantially real-time health data from patient device 18 to allow for a timely and effective intervention mechanisms.

Network system 10 may also include one or more healthcare provider systems 14 (e.g., PCP—or primary care provider systems). These provider systems 14 may periodically receive patient data from patient device 18, optionally as relayed through one or more servers. Medical practitioners may use provider systems 14 to diagnose patients, monitor effectiveness of therapy regimens, access EHR systems and facilitate similar functions or other tasks related to the provision of healthcare. Network system 10 may allow medical providers to monitor and diagnose many patients while simultaneously allowing designated family, friends, nurses, and the like to also monitor the patient's therapy regimen compliance, potential emergency and other situations or statuses.

Network system 10 may include or access cloud resources 15. Cloud resources (e.g., cloud service systems) 15 may provide a broad range of services, including using autonomous agents (for example, bots) for monitoring patient data received from patient device 18 and, at times, responding to patient data by sending a message or control signal to patient device 18 or contacting escalation services (e.g., “911”) according to rules defined by the applicable health service policy. Autonomous agents may provide continuous (i.e., 24/7) monitoring and may trigger appropriate responses in response to any type of event (these responses could include providing a message to a patient in response to a missed dose, contacting patients in response to a trending health incident, or contacting emergency services in the event of a detected emergency, for example). Cloud resources 15 may also include databases, dynamic resource registries, health service policies, monitoring services, data analytics, on-boarding and patient activation services, and the like.

A patient device 18 may also contain health service policy logic and algorithms configured to monitor continuously a patient's health events. In certain embodiments, local health service policy logic may trigger appropriate responses to any type of event.

Network system 10 may also illustrate a functional and communication relationship between many computing devices. However, each computing device's configurations may be governed by a patient-centered policy templates discussed below. In many embodiments, the patient-centric policy template provides a functional hierarchy and structure to the relationship of the computing devices making up network system 10, including providing escalation and intervention services and payer approval/preapproval services.

The devices and systems comprising network system 10 may be operatively coupled by one or more networks 12. Network 12 may include one or more of the Internet, local area networks (“LANs”), wide area networks (“WANs”), mobile telecommunications networks (e.g., 3G and 4G networks), and any suitable type of communication mediums. As illustrated in FIG. 1, the network system 10 may include one or more decentralized computing/communication devices or systems (14, 15, 16, 17, 18) configured to communicate with one or more other computing devices. For example, each of a plurality of computing devices may use mobile health client software executed on a computing device using a microprocessor, random access and fixed memory, input, and output devices, and facilitating common communication protocols such as Bluetooth, Wi-Fi and 4G and/or 5G data and voice access. Some configurations may capture and store patient data in a distributed fashion, for example, on the patient's computing device, rather than storing and accessing data on a central server or plurality of distributed servers.

Each communication device (16, 17, 18) in the decentralized network 12 may originate communications to any other communication device in the decentralized network. Thus, the decentralized network may allow for scaling of compliance and intervention tasks. For example, upon detection of a compliance event, the patient device 18 or a connected device/server may determine if the detected compliance event should be escalated to an incident such that a notification or alert should be sent to a health professional or patient representative. Thus, configurations may allow for a direct one-to-many communication notifying any of the system participants of a compliance event or other event or status. The embodiments also allow for many-to-one communications, for example, from health care providers or patient representatives to the patient. Certain configurations may also provide for direct communication between the patient communication device 18 and intervention services if the there is a determination that that level of escalation is desired, for example a healthcare professional or even 911.

In addition to the patient side solutions for the patient and his/her representatives, some configurations of the disclosure may employ a decentralized patient network operations console (not shown in FIG. 1) for healthcare providers as well as for the non-professional responsible for managing multiple patients. A console/dashboard may be dynamic and driven by functional role, permissions and availability of resource enabling real-time monitoring, intervention, and mobilization of resources in response to any health event/status/escalation. This type of console functionality may enable a healthcare professional (physician, nurse, nurse practitioner, nutritionist, an allied health professional, and/or a health coach) to have a personalized dashboard specific to their role and expertise so they may provide continuity of care services concurrently to a number of members each with their own discrete, individually tailored physician directed parameters of therapeutic care.

Configurations contemplate the use of sensors that determine vitals, wellness, or other health-relevant information about a patient. Examples of these types of sensors are thermometers, scales, blood oxygen sensors, blood pressure cuffs, and similar devices that provide telemetry/biotelemetry readings or any therapeutically relevant status or reading related to a patient. The patient's computing device may operate as a sensor or may receive data from the sensors in real-time, near real-time, periodically, or event-based, when a sensor reading or measured trend passes a threshold, etc. Certain configurations may use standard (e.g., cross-platform (e.g., iOS, Android, BlackBerry OS, Windows Phone 7, etc.)) plug-ins to interact with devices. Additionally, configurations may be deployed using existing or to-be-developed computing systems and communication platforms.

Medical-related organizations may be onboarded to configurations of the system either by invitation from the system administrator or by enrolling in a program associated with the system. Patients may be onboarded in any manner for onboarding individuals to software resources. For example, they may be onboarded to the network system 10 and become network participants using a link or invitation provided, for example, by a healthcare provider that already is a participant. A patient representative may be similarly onboarded or alternatively receive a link or invite from the patient and facilitated by the patient's mobile application. Patient representatives may be designated with one or more particular roles, thereby defining their capabilities in the system.

Configurations of the disclosure contemplate the handling of patient data consistent with government regulations (e.g., HIPPA) and patient privacy expectations. In most configurations, all patient health information (PHI) is protected by both access restrictions and encryption. For certain configurations, the access limitations are determined by the patient's permissions and/or by an associated healthcare provider. Encryption or other data protection techniques are employed relative to best practices and expected to evolve over time.

In some configurations, all data in the system is maintained in one or more logical location, such as a database, designated or agreed by the system administrator. Alternatively, more sensitive data may be maintained closer to and in storage controlled by the data owner (e.g., healthcare provider or patient), with less sensitive data flowing to a centralized logical database or server.

Configurations of the disclosure provide for access to data being designated based upon one or more of a per datum basis, a per patient basis, a per provider basis, a per publisher basis, a per subscriber basis or otherwise. In one or more configurations, multiple entities may have access to the patient data or portions thereof. For example, for a certain patient policy template, the patient data may be shared with multiple providers engaged in the treatment of the patient; perhaps a primary care physician, a medical oncologist, a surgeon, and a physical therapist. In some configurations, entities that are not providers may also have access to the data or portions thereof, such as family members, caregivers, insurers, researchers, system developers, health networks, institutions, or organizations.

To improve performance and meet quality and cost targets, providers and payers leverage technology disclosed herein, including distributed ledgers and remote patient care systems, to get ahead of clinical risks, to engage patients, and to effectively optimize healthcare costs and revenue cycle. Therapeutic evidence of activities of a patient's care at home (tolerance, adherence, and risk) is made available to payers and providers for them to satisfy value-based contracts. To do this, a proof of value system as disclosed herein is used in the health information network.

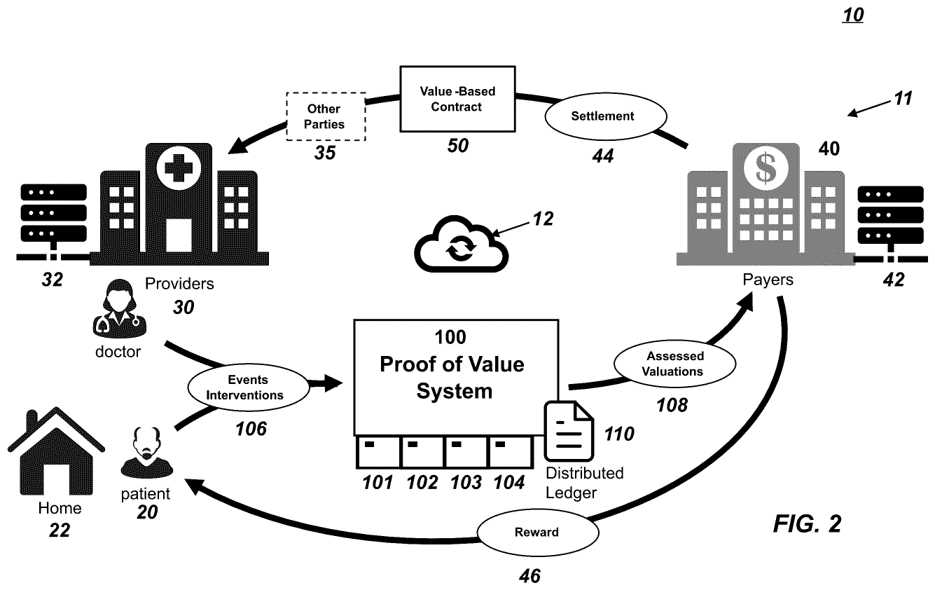

FIG. 2 illustrates a proof of value (PoV) system 100 utilized in a health information network system 10. The PoV system 100 works with network nodes 11 over one or more networks 12. The network nodes 11 are associated with members in the healthcare environment, including, for example, payers 40 having payer systems or “node” 42, health providers 30 having provider systems or “node” 32, and patients 20 having patient systems or “node” 22.

Additionally, other parties 35 may be included in the network of nodes as necessary to deliver care. For instance, a Beneficiary and Family Center Care (BFCC) organization may deliver care management services to patients and family plus coordinate the delivery of community resources to aid the patient's care. The PoV system 100 is inclusive of all care-related interactions between patients and professionals.

The PoV system 100 uses the decentralized health information network system 10 that spans distributed data sources and systems 22, 32, 42, even extending into patients' homes. In that sense, the PoV system 100 can be a cloud-based system or can be a decentralized or distributed system that utilizes and/or is spread across the distributed data sources and systems 22, 32, 42. As used herein, the PoV system 100 may be referenced as a distributed network system, a distributed system, or simply the “disclosed system.”

The distributed network system 100 coordinates a proof-of-value consensus governed by a value-based consensus contract 50 between payers 40 and providers 30 for patients 20). Briefly stated, the PoV system 100 receives interventions or actions from patients 20 and providers 30, determines the assessed value of those interventions, and provides the assessed value to payers 40. Based on the assessed value, the payers 40 can provide an appropriate settlement 44 according to the value-based consensus contract 50 to the providers 30. Additionally, the payers 40 can provide an appropriate reward to the patients 20 based on the assessed value.

In the present disclosure, references are made to an “intervention” or an “action.” These can be used interchangeably. In customary understanding, an intervention may refer to a medical-related action, but that is not strictly the case according to the context of the present disclosure. As disclosed herein, for instance, an “intervention” or an “action” in response to a patient's diagnosis can include any of the customary, medical-related actions. Additionally, an “intervention” or an “action” in response to a patient's diagnosis can include additional practices, such as, for example, assigning the patient to a care plan for that condition and can further include assigning a follow up call in a designated time period (e.g., one week) to be made from the care manager to assist the patient. Additionally, plans for interventions and actions according to the present disclosure may be multi-fold and may be conducted in parallel.

In general, the disclosed system 100 includes a therapeutic module 101, a correlation module 102, a rules engine or rules 103, and a distributed ledger or blockchain module 104, which uses a Proof of Value consensus protocol to capture direct (unrepudiated) evidence that patient interactions (interventions or actions 106) have been completed. In turn, the Proof of Value consensus protocol of the disclosed system 100 attributes predefined valuations 108 for each evidenced intervention 106.

Each intervention or actions 106 of a medical-related, therapeutic-related, or other type of event for the PoV consensus protocol is uniquely identified in a distributed ledger 110 of the disclosed system 100. The distributed ledger 110 includes information about an origin (i.e., physician/diagnosis or patient/therapeutic escalation), a mediator (i.e., patient/care plan acceptance or physician/prescription), and validators (i.e., health plan administrator, healthcare provider, and health consumer/patient). The distributed ledger 110 also attributes measured valuations surrounding the performance, the health quality, and the outcomes for the therapeutic events (e.g., medically related events, educating, literacy, caregiving, etc.) and interventions or actions 106. The valuations 108 are governed by the consensus contract 50 between the payers 40 and providers 30. In addition to providing the distributed ledger 110 for the assessed valuations 108 of the event interventions, the disclosed system 100 acts as a therapeutic event aggregator, which has a number of uses on its own.

At a minimum, the distributed ledger 110 is preferably protected, having restricted read/write access privileges to the information contained therein. Additionally, the distributed ledger 110 is also preferably self-proving, indicating the source of information, the history of the information, and the changes to the information. More particularly, the distributed ledger 110 is a blockchain using a decentralized, distributed ledger technology. In that sense, the distributed ledger 110 can have a continuously growing list of records, called blocks, which are linked together and secured cryptographically.

The distributed ledger 110 is decentralized so that the devices, systems, and other “nodes” 11 in the decentralized healthcare network system 10 can operate on the ledger 110. Each node 11 has a copy of the entire blockchain, and no single entity has full control over it. This decentralized nature enhances transparency, security, and resilience.

For a given patient, a new distributed ledger 110 can be constructed for each medical-related event, diagnosis, or other discrete element and may contain a record of all transactions that have ever occurred on the network related to that discrete element. Alternatively, for a given patient, the distributed ledger 110 can be persistent and may contain a record of all transactions that have ever occurred on the network. Either way, these transactions are grouped into blocks, and each block is linked to the previous one, forming a chain.

Every node 11 on the network system 10 has a copy of this ledger 110, ensuring that the information is distributed and replicated across the network system 10. Due to the existence of medical information contained, the distributed ledger can preferably use a permissioned blockchain technology, which restricts access to authorized participants. Permissionless blockchain technology may be used in some circumstances.

The data recorded in a block and added to the distributed ledger 110 is secured from alteration or tampering by using cryptographic hashing. In this technique, each block contains a unique cryptographic hash of the previous block. Any attempt to alter a given block requires changing all subsequent blocks, which is computationally infeasible and would require consensus from the majority of nodes 11 in the network system 10.

As disclosed herein, the decentralized health information network system 10 disclosed herein employs a consensus mechanism (i.e., Proof of Value (PoV)) to validate and agree on the state of the distributed ledger 110. The consensus mechanism determines how new blocks are added to the ledger 110 and defines how conflicts are resolved. These mechanisms ensure that all nodes 11 in the network system 10 reach agreement on the validity of transactions without the need for a central authority to resolve conflicts.

As disclosed herein, the decentralized health information network system 10 disclosed herein supports self-executing “smart” contracts-i.e., the consensus contract(s) 50 described herein. Accordingly, the contract terms are directly written into code used in the network system 10 so the code can automatically enforce and execute the contract terms of the agreement once predefined conditions have been met. By having these features, the disclosed system 100 can use digital decentralized applications (DApps), which can run on the network system 10 of nodes 11.

The PoV consensus protocol of the disclosed system 100 is completed once “proof of stake/action” is registered in the network system 10—i.e., by a healthcare provider 30 (e.g., a coded encounter, a referral, and/or a prescription) and/or by a health consumer (e.g., a patient 20 or a patient's authorized proxy who may schedule appointments and/or may take certain therapeutic actions). In the end, to achieve Proof of Value consensus, a given medical-related event intervention requires consensus among all three stakeholders (e.g., payer 40, provider 30, and patient 20 (or patient proxy)).

Using the disclosed system 100, for example, a provider 30 of record can access the therapeutic module 101 and can prescribe a digital therapeutic for a diagnosed condition for a patient 20. The correlation module 102 of the disclosed system 100 performs a correlation to identify a medical-related event, and the rules 103 of the rules engine integrates policy-based care management at the point of care, such as in the patient's home. The therapeutic module 101 and rules 103 enable early signal detection of patient health status and deterioration. The rules 103 of the engine preferably satisfies CMS performance and preferably makes quality measurements associated with the value-based consensus contract 50. To do this, the disclosed system 100 provides data related to health status and therapeutic adherence and tolerance between clinical encounters, and the disclosed system 100 quantifies the impact of earlier (lower cost) health interventions.

The ledger module 104 constructs a distributed ledger 110 having the condition identification, including the provider 30, patient 20, and the condition and having other information (e.g., intervention or action 106 and the assessed valuation 108) for the proof of value discussed herein.

This decentralized health information network system 10 can take feeds from provider RCM systems, pharmacy claims systems, payer risk stratification solutions, as well as the remote care management and monitoring solutions in the home. This decentralization enables unique digital pathways for micro-interventions (adjustments in care) as well as value attribution based upon the PoV consensus of the provider 30, patient 20, and payer 30 for a “therapeutic course of action”.

The technology disclosed herein makes it possible for a patient to record observations, such as their symptoms, vitals, medication use, and the like, on a frequent basis, even daily. The patient can share this information with their physicians using the remote patient monitoring capabilities of the disclosed system 100. In turn, the disclosed system 100 can also communicate interventions to the patient, healthcare providers, and others based on the patient's observation. For example, the disclosed system 100 can alert the patient to contact their care team when severe symptoms are present and physical medical intervention is required.

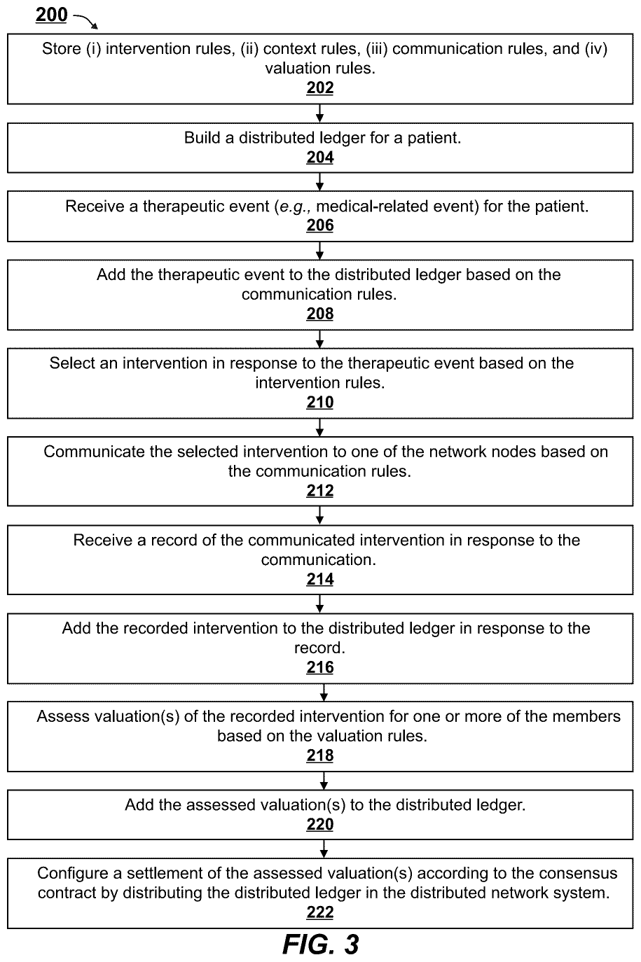

FIG. 3 illustrates a flowchart of a process 200 according to an example of the present disclosure to fulfill proof of value transactions according to a consensus contract. In this example, one or more process blocks of FIG. 3 may be performed by the PoV system (e.g., distributed network system) 100 on the network system 10 discussed above. For further understanding, reference to elements in FIGS. 1-2 may be used in the discussion of the disclosed process 200.

In the disclosed system 100 involving various network nodes 11, proof of value (PoV) transactions are fulfilled according to a consensus contract 50 using the disclosed process 200. The PoV transactions start with the medical-related events for patients 20 and encompass the interventions or actions 106, assess valuations 108, and settlements/rewards 44, 46.

As noted above, the network nodes 11 are at least associated with patients 20, providers 30, and payers 40. The disclosed system 100 operates in various healthcare environments involving various network nodes 11 and medical-related events, ultimately ensuring seamless fulfillment of PoV transactions (106, 108, 44, 46) according to the established consensus contract 50.

Briefly, the providers 30 can include one or more of a healthcare provider, a hospital, a doctor, a medical practitioner, a medical supplier, a pharmacy, etc. The payers 40 can include one or more of a health management company, a medical insurer, an insurance company, a health plan administrator, etc. The medical-related events can include one or more of a medical diagnosis, a medical observation, a patient's biotelemetry data, etc. Meanwhile, the interventions can include one or more of a therapeutic protocol, a digital therapeutic program, a directed action, a communication from a care navigator or social worker, a reward for achieving a therapeutic goal, an increase in care giving support in the home, providing medical equipment to improve monitoring, an adjustment to a patient care plan (i.e., plan's scope, schedule, or thresholds for intervention), etc.

The process 200 involves several steps, all orchestrated by the disclosed system 100. Initially, the disclosed system 100 stores a multitude of rules 103—intervention rules, context rules, communication rules, and valuation rules (Block 202). The intervention rules, communication rules, and valuation rules are governed by a PoV consensus protocol from the consensus contract 50. The context rules, which are discussed in more detail below, define context indicators, which are determined by analysis of factual circumstances associated with the patients. As will be appreciated, the rules 103 are stored in one or more databases of the disclosed system 100. These rules 103 define specific interventions for defined medical-related events, attribute valuations to the members (e.g., patients, providers, etc.) in the healthcare environment for these interventions, and outline communication protocols for interacting with each network node 11 for the interventions.

For example, the intervention or action rules are used to detect and select specific interventions or actions in response to defined events, such as medical-related events or other types of events. The intervention or action rules can generally define how each event (e.g., a medical-related event, a therapeutic-related event, or other type of event) is detected and how any one or more specific interventions or actions are subsequently selected. As noted above, reference to “intervention” and “action” may be used interchangeably herein. Additionally, the “event” referenced herein need not be “medical-related” only, as other types of events may be involved. For instance, the hospitalization of a spouse giving care to the patient can constitute an “event” (e.g., a “therapeutic-related event”) according to the present disclosure. The intervention or action rules are used to detect and select specific intervention(s) or action(s) in response to such defined therapeutic-related events. The rules can generally define how each therapeutic-related event is detected and how any one or more specific actions are subsequently selected. For example, the therapeutic-related event involving the hospitalization of a spouse giving care to the patient may involve a plan of action(s) including scheduling an additional communication from the health plan care manager and assigning a part-time caregiver to assist the patient. The valuation rules can generally assess recorded interventions by defining how the recorded interventions or actions are detected and how the recorded interventions or actions are subsequently valued. The intervention or action and valuation rules make up a form of business logic implemented at the disclosed system 100.

For their part, the communication rules generally govern how the disclosed system 100 interfaces with network nodes 11, such as defining how the disclosed system 100 sends and receives communications. In that sense, the communication rules are engagement and collection rules defining how the disclosed system 100 engages with network nodes 11 and how the disclosed system 100 collects information related to medical-related events and inventions from the network nodes 11 or from elsewhere.

During the process 200, a distributed ledger 110 for a given patient 20 is constructed using one or more processors within the disclosed system 100 (Block 204). Building the distributed ledger 110 starts with receiving an event (e.g., a medical-related event or other type) for the patient 20 (Block 206) and then adding or incorporating the event into the distributed ledger 110 according to the communication rules (Block 208). The event can be received from any number of sources, such as from any one of the network nodes 11. As a few examples, a medical-related event can come in the form of a diagnosis from a provider 30, an observation from the patient 20 or patient representative, or biotelemetry data from a device at the patient's home. As another example, a therapeutic-related event can come from the payer (40) detecting the hospitalization of a spouse that was providing caregiving to the patient.

An appropriate intervention or action 106 for the medical-related or other type of event is selected based on the intervention rules (Block 210). The intervention or action 106 is then communicated to the relevant network node(s) 11 based on the communication rules (Block 212). The intervention or action 106 can be communicated to any appropriate one or more of the network nodes 11 depending on the intervention. For example, some forms of intervention or action 106, such as a digital therapeutic program, can be communicated directly to the patient 20, while other forms of intervention 106 may be communicated to other network nodes 11 or other destination.

When a record of the communicated intervention or action 106 is then received at the disclosed system 100 (Block 214), information of the intervention or action 106 is added or incorporated into the distributed ledger 110 (Block 216). The record can take many forms, including an acknowledgement, a consent, or other indication directly from the recipient member's network node 11, indirectly from another member's network node 11 related to the particular intervention, or from another source.

The process 200 then involves evaluating the intervention's impact on involved member(s) (e.g., patient, provider, etc.) by assessing one or more given valuations 108 of the recorded intervention or action 106 for a given one or more of the members (e.g., patient, provider, etc.) based on the valuation rules (Block 218). Information about these assessed valuations 108 is then added to the distributed ledger 110 (Block 220).

The disclosed system 100 further ensures that a settlement 44 (or reward 46) of these assessed valuations 108 is fulfilled for the members (e.g., patient, provider, etc.) in accordance with the consensus contract 50 by distributing the ledger 110 across the network system 10 and configuring the settlement 44 (or reward 46) of the assessed valuations 108 accordingly (Block 222).

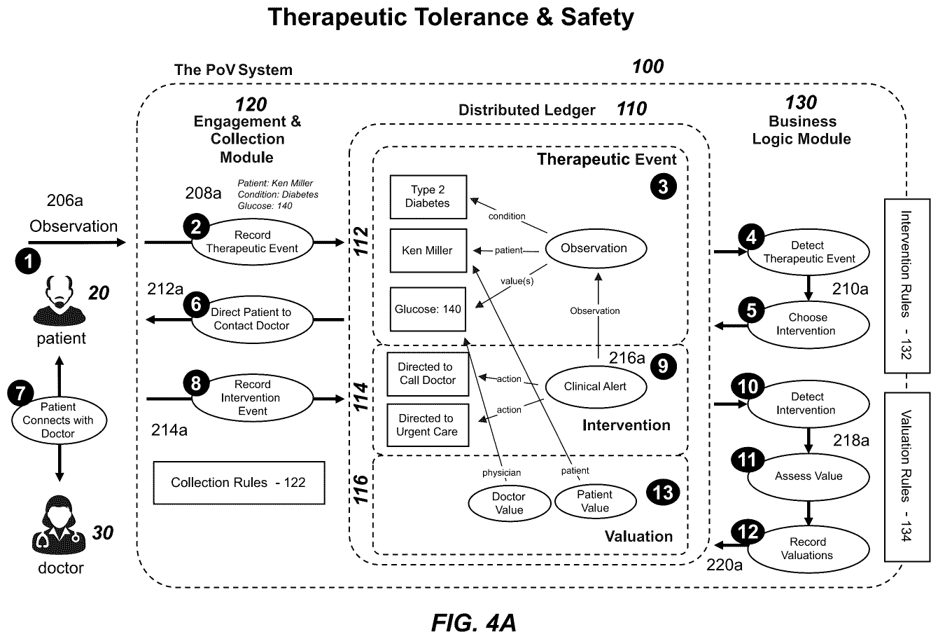

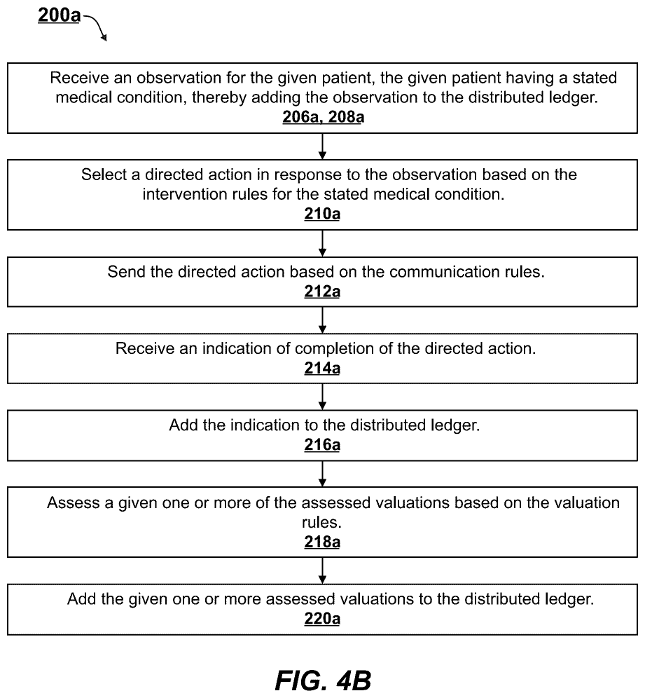

In specific instances, such as receiving diagnoses or observations for given patients 20, the disclosed system 100 selects interventions or action 106, communicates them to the relevant network node(s) 11, records their completion, and assesses valuations 108 based on the intervention rules and the valuation rules outlined earlier. FIGS. 4A-4B described below show an example. Additional details can be found in incorporated U.S. Prov. Appl. ______ filed herewith and entitled “System and Method for Therapeutic Event Management and Value Attribution (Proof of Value) in Decentralized Health Information Network” with Attorney Docket number 266-0006PUS.

The process 200 may include additional implementations, such as any single implementation or any combination of implementations described below and/or in connection with one or more other processes described elsewhere herein.

Although FIG. 3 shows example blocks of the process 200, the process 200 in some implementations may include additional blocks, fewer blocks, different blocks, or differently arranged blocks than those depicted in FIG. 3. Additionally, or alternatively, two or more of the blocks of the process 200 may be performed in parallel.

FIG. 4A illustrates an example of the PoV system (e.g., distributed network system) 100 in more detail. Here, the disclosed system 100 is being used for therapeutic tolerance and safety of a patient 20. For further understanding, reference is also made to FIG. 4B, which illustrates a portion of the PoV process 200a for the therapeutic tolerance and safety example in FIG. 4A.

The disclosed system 100 uses an engagement and collection module 120 (having the disclosed communication rules 122) and uses a business logic module 130 (having the intervention rules 132 and valuation rules 134) to select intervention, to perform valuations, and to construct a distributed ledger 110, which is then used throughout the disclosed system 100 and network system 10. When constructed, the distributed ledger 110 for a given patient 20 includes information about an underlying therapeutic event (e.g., medical-related event) (section 112), an intervention selected to address that therapeutic event (medical event) (section 114), and an assessed valuation for the intervention (section 116).

The engagement and collection module 120 uses the communication rules 122, which define how the disclosed system 100 interfaces with doctors 30 and patients 20, including how a therapeutic event (e.g., medical event) is recorded, how a therapeutic program is prescribed to the patient 20, and how the patient 20 consents to the program. The business logic module 130 uses the intervention rules 132, which define how therapeutic events (e.g., medical events) are detected and how interventions are in turn selected. Additionally, the business logic module 130 uses valuation rules 134, which define how an intervention is detected and how that intervention is then valued.

During therapeutic tolerance and safety, the patient 20 makes observations (symptoms, vitals, medication use, etc.) using the disclosed system 100 (Step 1). For example, the patient 20 may take glucose readings at home using a glucose monitor, and these glucose readings may be entered into a client device for processing. Alternatively, the glucose monitor may operate automatically as a client device capable of processing according to the disclosed techniques.

Using the communication rules 122, the engagement and collection module 120 records the observations (e.g., medical event) (Step 2) by adding it to the distributed ledger 110 (Step 3). In the provided example, the patient (Ken Miller) has observed a glucose level of 140 mg/dL, which is added to the therapeutic event section 112 of the distributed ledger 110.

The business logic module 130 of the disclosed system 100 now detects the therapeutic event (Step 4) and selects intervention(s) for the detected medical-related event (Step 5). Using the intervention rules 132 governing therapeutics available for the medical-related event (e.g., diagnosed condition), a set of interventions may be selected and initiated based on the type of medical-related event. In this example, the disclosed system 100 initiates the assignment of the patient 20 to a digital therapeutic program for diabetes. Like all intervention logic, metrics provided to the system 100 are compared to thresholds in the interventions rules 132 to determine the resulting intervention to be selected. The disclosed system 100 then matches the candidate criteria to a specific therapeutic for the resulting intervention.

Before the selected intervention(s) are actually added to the distributed ledger 110, the engagement and collection module 120 interacts with the patient 20. In particular, the disclosed system 100 prescribes the digital therapeutic program, which is communicated to the patient 20 for acceptance (Step 6). The system 100 obtains the patient's acceptance (consent) to the therapeutic program (Step 7). In turn, the system 100 records the engagement event (Step 8) and records the intervention into the intervention section 114 of the distributed ledger 110 (Step 9).

In response to the therapeutic engagement by the patient 20, the business logic module 130 now detects the therapeutic event (Step 4) and selects intervention(s) for the detected medical-related event (Step 5). Using the intervention rules 132 governing therapeutics available for the medical-related event (e.g., observation), a set of interventions may be selected and initiated based upon the type of medical-related event. In this example, the disclosed system 100 initiates a clinical alert intervention (i.e., a directed action) directing the patient 20 to contact the doctor 30 immediately (Step 6). Like all intervention logic, metrics provided to the system 100 are compared to thresholds in the intervention rules 132 to determine the resulting intervention event. In this case, the patient's glucose level exceeded a safe threshold, requiring the directed action.

In response to the indication of contact, the business logic module 120 now detects the intervention (Step 10), assesses the value of the intervention (Step 11), and records the valuation(s) into the distributed ledger 110 (Step 12). The valuations are added to the valuation section 116 of the ledger 110 and include physician value and patient value (Step 13). Based on the type of intervention, the value of the intervention is assessed (Step 11) for all parties involved. Like all assessment logic, metrics are compared to thresholds in the valuation rules 134 to determine the resulting values assigned. For instance, the value of clinical alert engagement can be scored based on the patient's and doctor's performance metric (e.g., time to respond, value of intervention in diminishing the risk of a more serious condition later, etc.). These metrics are assessed against the contract thresholds of the value-based consensus contract 50.

In the counterpart process 200b in the flowchart of FIG. 4B, the distributed network system 100 receives the given medical-related event for the patient 20, which involves obtaining an observation of the patient 20 who has a specified medical condition (e.g., type 2 diabetes) (Block 206b), thereby incorporating the observation related to the stated medical condition into the distributed ledger 110 (Block 208b).

The distributed network system 100 selects a directed action for the patient 20 in response to the observation based on the intervention rules 132 for the specified medical condition (Block 210b). The disclosed system 100 then communicates this directed action to the patient 20 according to the communication rules 122 (Block 212b). Upon receiving an indication of completion of the directed action (Block 214b), the disclosed system 100 records this completion and adds the indicated completion to the distributed ledger 110 (Block 216b). As will be appreciated, the directed action can include any number of actions to be performed by the patient depending on the stated medical condition and the reported observations.

Subsequently, the distributed network system 100 assesses one or more of the evaluated valuations for both the patient 20 and the provider 30 based on the valuation rules 134 (Block 218b). Finally, the disclosed system 100 adds these assessed valuations for both entities to the distributed ledger 110 (Block 220b).

The solution disclosed here is directed to patients who are engaged in a remote patient monitoring program, where the remote patient monitoring program regularly captures patients' information. Additionally, alerts prompt the patients to engage their care team between clinical visits when appropriate, which forms a single type of therapeutic micro-intervention. Several other types of therapeutic micro-interventions related to consensus protocols are possible, including: therapeutic surveillance (consensus of care), therapeutic engagement (consensus of intervention), therapeutic navigation (consensus of education), therapeutic tolerance (consensus of efficacy), therapeutic adherence (consensus of care—patients), therapeutic safety (consensus of safety), therapeutic risk (consensus of escalation and intervention), therapeutic reward (consensus of achievement), therapeutic supply chain (consensus of fulfillment), therapeutic adherence, therapeutic tolerance (side effects of therapeutic), therapeutic safety (symptoms and vitals), and therapeutic supply chain and fulfillment. Moreover, therapeutic micro-interventions can also be assessed based on demographic information, patience resilience, and social determinants of the patients. One such micro-intervention includes a directed action for a patient to engage a provider.

As discussed above, the Proof of Value (PoV) system 100 captures evidence of contributions of all three players (e.g., patient, provider, and payor). These evidence streams from the disclosed system 100 are combined with additional sources of information about the patient's context, including the patient's personal resiliency (i.e., patient resilience), the patient's Social Determinants of Health (SDoH) risk profile, and the extent of the patient's caregiving network. As will be appreciated, the patient's SDOH risk profile can include a number of relevant factors, including housing, food and nutrition, transportation, social and economic mobility, education, and environmental conditions. Health-related social needs (HSRNs) refer to the patient's needs that might include affordable housing, healthy foods, or transportation. The patient resilience relates to the psychological and emotional aspects of the patient.

As used herein, for example, the patient resilience refers to the ability of the patient to withstand, cope with, and recover from physical or mental health challenges, adversity, or stressors. Overall, resilience is not defined by a fixed trait and is instead defined by a dynamic determination influenced by numerous factors, including genetics, environment, social support, and personal characteristics.

Resilience plays a crucial role in the management and outcomes of chronic diseases, acute illnesses, and traumatic injuries. Resilient patients demonstrate adaptability and resourcefulness in the face of illness, injury, or other health-related setbacks. For that reason, resilient patients are better equipped to bounce back from setbacks, maintain a positive outlook, and continue pursuing goals despite obstacles.

The disclosed system 100 assesses the patient resilience to produce any suitable context indicators as part of a comprehensive care planning and treatment strategy. Understanding the patient's resilience can help the disclosed system 100 tailor interventions to support the patient's coping mechanisms, strengthen the patient's resilience factors, and enhance the patient's overall well-being.

The disclosed system 100 determines an optimal intervention strategy based upon the patient's profile and context. From the anticipated value of each potential intervention, an optimal intervention plan is selected, and resources assigned. For instance, if a patient is isolated with little support after a serious therapeutic event, a regular call from their payor's care manager may have the highest value to improve the patient's outcome by lifting the spirits. In another instance, for a patient who has had a serious therapeutic event and who has a wide care giving network of family, an alternative intervention may be most appropriate if the patient has financial hurdles.

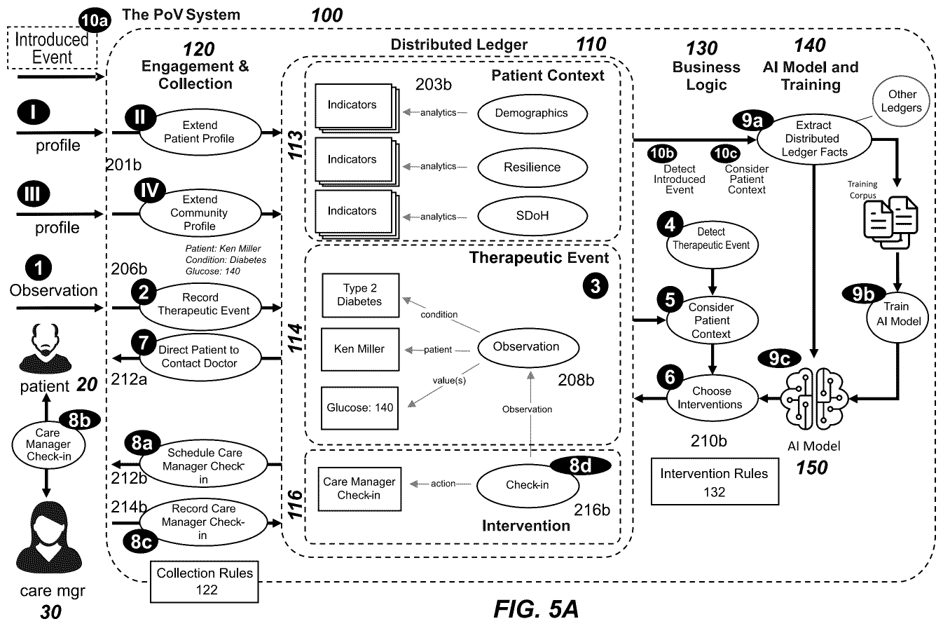

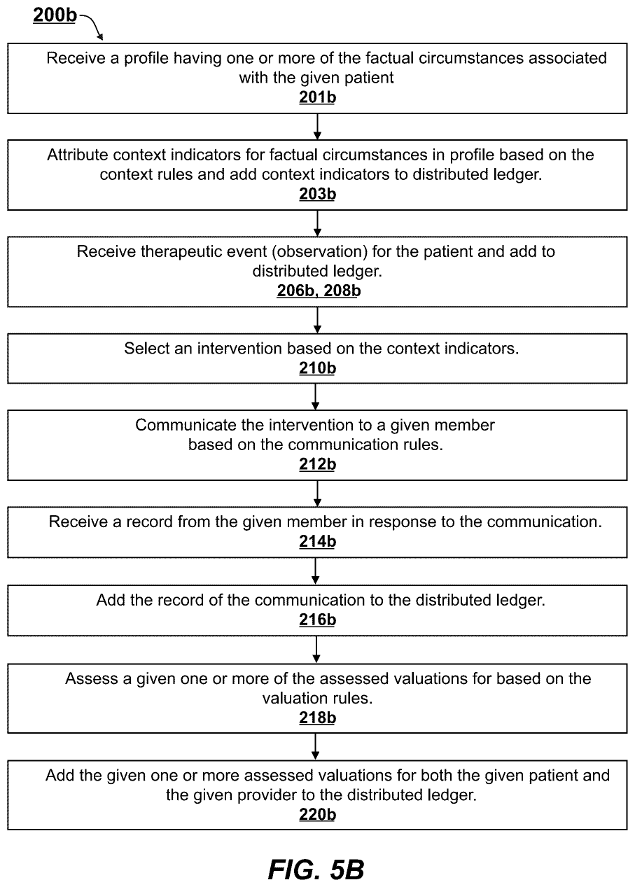

FIG. 5A illustrates an example of the disclosed system 100 being used for therapeutic micro-intervention according to the present disclosure. For further understanding, reference is also made to FIG. 5B, which illustrates a portion of the PoV process 200b for the therapeutic tolerance and safety example in FIG. 5A.

Initially, the PoV system 100 obtains contextual information (i.e., factual circumstances) about the patient so that optimal intervention strategies can be selected. In particular, the disclosed system 100 obtains the patient profile information and community profile information (Steps I, III) and adds the extended profile information to a patient context section or block 113 of the distributed ledger 110 (Steps II, IV). These steps can capture: the patient's demographic information; individual resilience and that of the care network; and the community's social determinants of health (SDoH). Other profile information can be included in these steps. The PoV system 100 uses the information to formulate a strong context for the patient.

Analytics are performed by the disclosed system 100 on the demographic factors, resilience factors, and SDoH factors of the patient's context to provide different contextual valuations or indicators. For example, the disclosed system 100 uses analytics to review information, such as the patient's income bracket; education level; and the patient's level of isolation to form strong valuations (e.g., predictors) for selecting better intervention strategies for the patient 30. The indicators can include values, weights, and other variables indicative of the factual circumstances of the patient and the patient's community.

During operation, the patient 20 and providers 30 interact with the PoV system 100 with several types of events, including a medical-related event, a therapeutic-related event, a diagnosis, an observation, a consumption of a medical supply, hospitalization of a care-giver, and a change to a patient's circumstances like financial, housing, or food security, etc. Based on the type of events, the PoV system 100 can provide a set of interventions or actions. To select the best intervention strategy, the PoV system 100 uses the patient context 113 and brings the information into the algorithms that formulate a course of action.

An example event shown in FIG. 5A includes a medical-related observation made by the patient. As will be appreciated, the PoV system 100 can be used with any number of medical-related events or other types of events.

During therapeutic tolerance and safety, for example, the patient 20 makes observations using the disclosed system 100 (Step 1). For example, the patient 20 may take glucose readings at home using a glucose monitor, and these glucose readings may be entered into a client device for processing. Alternatively, the glucose monitor may operate automatically as a client device capable of processing according to the disclosed techniques.

Using the communication rules 122, the engagement and collection module 120 records the observations (e.g., medical event) (Step 2) by adding it to the distributed ledger 110 (Step 3). In the provided example, the patient (Ken Miller) has observed a glucose level of 140 mg/dL, which is added to the therapeutic event section 112 of the distributed ledger 110.

The business logic module 130 now detects the medical event (Step 4) and considers the patient's context (i.e., factual circumstances related to housing, food and nutrition, transportation, social and economic mobility, education, environmental conditions, concurrent symptoms, past observations, and trends, etc.) (Step 5) and selects intervention(s) for the detected medical-related event (i.e., observation) (Step 6).

To select the best intervention strategy, the PoV system 100 uses the patient context 113 (Step 5) and brings this information into the algorithm that formulates the course of action (Step 6).

In this example, the PoV system 100 initiates (Step 7) a clinical alert intervention directing the patient to contact his doctor immediately. The clinical alert is determined based on the glucose level exceeding a threshold.

Using the intervention rules 132 governing therapeutics available for the medical-related event (e.g., observation), a set of interventions may be selected and initiated based upon the type of medical-related event. In this example, the disclosed system 100 initiates a clinical alert intervention (i.e., a directed action) directing the patient 20 to contact the doctor 30 immediately (Step 6). Like all intervention logic, metrics provided to the system 100 are compared to thresholds in the intervention rules 132 to determine the resulting intervention event. In this case, the patient's glucose level exceeded a safe threshold, requiring the directed action.

Before the selected intervention(s) are added to the distributed ledger 110, the engagement and collection module 120 interacts with the patient 20. In particular, the disclosed system 100 directs the patient 20 to contact his doctor 30 (Step 7).

In the present example of FIG. 5A, the patient's context may also warrant additional interventions (i.e., auxiliary, secondary, or micro-interventions) to be made. For example, the patient 20 may live alone and his condition may have been worsening over time. These circumstances trigger an additional intervention to be performed. The interventions disclosed herein may therefore be aimed at fostering the patient's resilience and can include psychosocial support, education, counseling, stress management techniques, and lifestyle modifications. For example, the intervention proposed in this example can include a directed action for a care manager to check-in with the patient within a time period (e.g., the next day).

In this auxiliary intervention, a check-in of the patient is scheduled for the care manager (Steps 8a-8d). A communication is sent to the care manager 30 to perform the check-in of the patient 20 (Step 8a). When the care manager 30 makes that scheduled check-in (Step 8b), the check-in contact is recorded by the disclosed system 100 (Step 8c) and is added to the intervention section 114 of the distributed ledger 110 (Step 8d).

The selection of the interventions (and especially the auxiliary interventions based on the patient's context disclosed herein in Step 6) can be assisted by an artificial intelligence (AI) model 150, which can be part of an AI module 140 of the disclosed system 100. As shown herein, the AI module 140 can extract the indicators and other contextual information from the patient context 113 and can feed this information into a trained AI model 150 (Step 9c) to select the intervention(s) (and especially the auxiliary interventions based on the patient's context disclosed herein in Step 6).

The AI model 150 of the AI module 140 can be trained in a training procedure (Step 9b). As briefly shown here, the AI module 140 can extract facts (i.e., factual circumstances, indicators, and the like) from the distributed ledger 110 of this patient and from other sources, such as from the ledgers of other patients. This data forms a training corpus to train the AI model 150 in a training procedure.

In this way, the AI model 150 used for selecting the interventions is trained in a training process (9b) using facts extracted from the distributed ledgers 110 and other sources. The training can train the AI model 150 to produce population level actions and interventions (A) using underlying indicators within varying patient contexts, conditions, therapeutic programs, and professional care. For a given patient having a medical-related event, the trained AI model 150 then detects the indicators for the given patient that indicate that a specific intervention strategy is needed.

The AI model 150 can score the additional intervention paths based upon anticipated likelihood/value of improving health outcomes and cost, thus determining the best strategy.

In this example, the AI model 150 determines the rationale for contacting the at-risk, isolated patient 20 who just experienced a therapeutic event (i.e., medical event). The AI model 150 presents a formulation of the rationale for contact as part of the contact list for the patient's care manager 30. Details related to training the AI model are further disclosed in incorporated U.S. Provisional Appl. No. ______ and entitled “System and Method for Protecting Patients' Therapeutic Treatment Context Used for Training Artificial Intelligence Systems” (Atty. Dkt. No. 266-0004PUS).

Additional operations of the disclosed system 100 can then be performed, such as disclosed above with reference to FIG. 4B. Once the patient 20 connects with the doctor 30 (Step 7) and/or the provider (e.g., care manager) connects with the patient (Step 8a), the disclosed system 100 obtains a record, acknowledgement, or other indication of the contact, which can come from the provider 30, and the disclosed system 100 in turn records the intervention event (Step 8c) into the intervention section 114 of the distributed ledger 110 (Step 8d).

In response to the indication, the disclosed system 100 can follow the steps related to FIG. 4A, which are not explicitly shown here. The business logic module 120 can detect the intervention (Step 10; FIG. 4A), assesses the value of the intervention in the consensus contract (50) (Step 11; FIG. 4A), and records the valuation(s) into the distributed ledger 110 (Step 12; FIG. 4A). The valuations are added to the valuation section 116 of the ledger 110 and can include physician value and patient value, for example (Step 13; FIG. 4A).

Additionally, the AI model 150 disclosed herein can be configured to detect more subtle scenarios and can indicate alternative courses of action. For instance, the AI model 150 can be trained to identify the side effects of a certain medication, prescribed by another doctor, such as the side effect on blood sugar. Drawing in the whole patient context (step 5), the AI model 150 can determine that a toxic or detrimental side effect is indicated and can indicate that the patient's providers be notified immediately. In this way, the intervention rules 132 can be informed by one or both of fixed thresholds and the knowledge base of the AI model 150, which can be selected individually or combined together to formulate appropriate actions for all members in the network system 10, including the patient 20, the provider 30, and the payer 40.

To further illustrate the network system 10, a member system (e.g., provider 30, other parties 35, etc.) may introduce an event that must be assessed to determine the implications the event can have upon the patient's health. Two examples are provided here. In a first scenario, the patient has been prescribed with a new medication. The event is presented to the distributed ledger 110 via a Pharmacy Benefit Manager acting in the network system 10 as one of the other parties 35. This new therapeutic event is assessed by a trained AI model 150 that can determine contraindication risk and can recommend an orchestrated intervention strategy. For example, the AI model 150 can recommend a directed action to inform the prescribing physician 30 of the contraindication risk and can recommend a directed action informing care managers 30 and the patient 20 and the pharmacy benefit management system 35 of the contraindication event.

A second scenario involves the death of a patient's caregiver. There are multiple means that this can be detected. For instance, the caregiver may be the patient's spouse and may be on common health insurance as the patient. Alternatively, the patient may reveal the death of their caregiver when sharing information with a physician or care coordinator 30. Once this event is presented to the distributed ledger 110, the AI model 150 can assess the implications of this event upon the patient 20 by taking into account the patient's context, such as reported resiliency, SDoH, acuity of health, etc. Based upon this assessment, the AI model 150 can formulate a recommended intervention strategy.

In the counterpart process 200b in the flowchart of FIG. 5B, the distributed network system 100 receives a profile having one or more factual circumstances associated with the patient (Block 201b). The disclosed system 100 then attributes context indicators (e.g., values, weights, etc.) for the patient's factual circumstances in profile based on the context rules and adds these context indicators to distributed ledger (Block 203b).

Once set up, a medical-related event may occur. For example, the patient may receive a diagnosis from a provider, the patient may make an observation (medical reading), etc. The distributed network system 100 receives the given medical-related event for the patient 20 (Block 206b). In this example, the given medical-related event involves obtaining an observation of the patient 20 who has a specified medical condition (e.g., type 2 diabetes) (Block 206b). The medical-related event is then added into the distributed ledger 110 (Block 208b).

At this point, the distributed network system 100 selects an intervention based on the context indicators (Block 210b). In this example, the distributed network system 100 selects a directed action for a provider (e.g., care manager) to contact the patient.

The disclosed system 100 then communicates this intervention to a given member based on the communication rules (Block 212b). In this example, the directed action to contact the patient 20 is communicated to the care manager according to the communication rules 122 (Block 212b). Additionally and as noted previously, the disclosed system 100 communicates a direct action to the patient to contact his doctor (i.e., Block 212a in FIG. 5B).

Upon receiving a record (e.g., an indication of completion of the directed action) (Block 214b), the disclosed system 100 records this completion and adds the indicated completion to the distributed ledger 110 (Block 216b). As will be appreciated, the directed action can include any number of actions to be performed by the patient, the provider, or other member depending on the medical-related event, reported observations the patient's stated medical condition, and the patient's factual circumstances affecting the patient's outcomes.

Subsequently, the distributed network system 100 assesses one or more of the evaluated valuations for the relevant member(s) (e.g., provider in this example) based on the valuation rules 134 (Block 218b). Finally, the disclosed system 100 adds these assessed valuations for both entities to the distributed ledger 110 (Block 220b).

Several types of AI models 150 can be used for the techniques of the present disclosure, including, for example, a symbolic AI model, a Bayesian network model, a machine learning model, a neural network model, and types of algorithmic models. A few of these models are briefly described.

As is known, a Bayesian network (a.k.a. a belief network or a causal probabilistic network) is a graphical model representing probabilistic relationships among a set of variables. The model includes nodes (representing variables) and includes directed edges (representing probabilistic dependencies) between the variables. At its core, the Bayesian network provides a joint probability distribution of the variables in a compact form by employing conditional independence relationships.

Each node in the Bayesian network represents a random variable and is associated with a conditional probability distribution that quantifies the probability of that variable given its parent variables. The parent variables of a node are the variables that directly influence the node's probability distribution. By specifying these conditional probabilities for each node, the Bayesian network encodes the entire joint probability distribution of the variables.

For their part, the directed edges in the Bayesian network define causal relationships or dependencies among variables. The direction of the edges indicates the direction of influence: from parent variables to child variables. This structure enables efficient inference by allowing reasoning about the probabilities of variables given observed evidence or interventions on certain variables.

A symbolic AI model uses explicit rules and logical reasoning to solve problems. In the symbolic AI model, knowledge is represented in a structured format, such as rules or ontologies, and algorithms manipulate these rules to derive conclusions or make decisions. The knowledge can be encoded as rules or if-then statements to provide recommendations or solutions.

A machine learning model learns from data to improve performance over time without being explicitly programmed. Several subtypes, including supervised learning, unsupervised learning, and reinforcement learning, can be used. Supervised learning involves training a model using labeled data, where the input-output relationships are explicitly provided. Supervised learning is suitable for classification and regression types of tasks. Unsupervised learning trains models using unlabeled data to discover hidden patterns or structures within the data and is suitable for clustering and dimensionality reduction. In reinforcement learning, an agent learns to make sequential decisions by interacting with an environment and receiving feedback in the form of rewards or penalties.

A neural network model has interconnected nodes, or neurons, organized into layers. Each neuron applies a mathematical operation to its inputs and passes the result to the next layer. Deep learning as a subset of a neural network trains models with multiple layers in a deep architecture to learn hierarchical representations of the data. Convolutional neural networks (CNNs) are suitable for image recognition and computer vision, whereas recurrent neural networks (RNNs) are suited to analyzing sequential tasks, such as natural language processing and time series prediction.

An algorithmic model iteratively evolves candidate solutions using mechanisms such as mutation, crossover, and selection. The algorithmic model is useful for problems having large search spaces or complex, multi-dimensional objective functions.

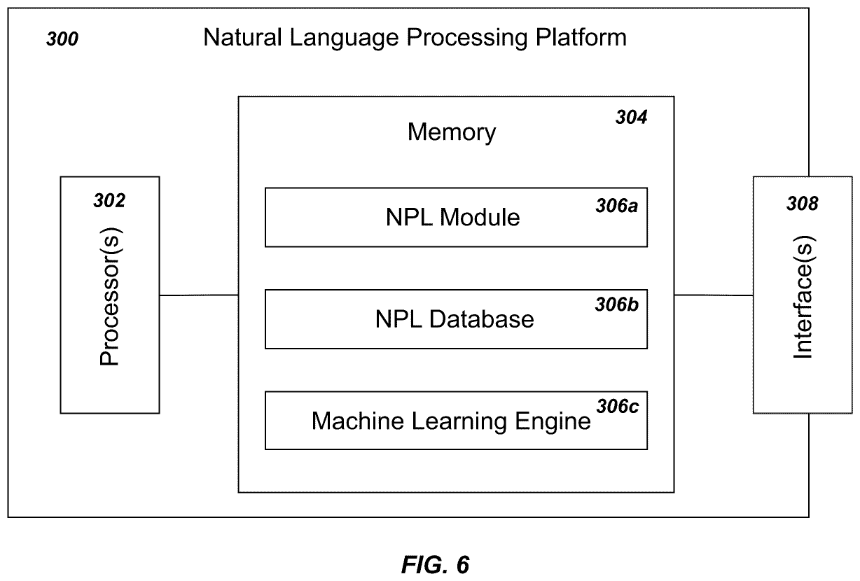

As one example according to the present disclosure, FIG. 6 illustrates a natural language processing platform 300 for preparing training data to be used in training an AI model in a computing environment. The NLP platform 300 can use large language models (LLMs) to enable a computing system to understand, process, and generate language and perform tasks that construct, arrange, and manage medical information to be used as training data for AI models. In general, the NLP platform 300 can be associated with an organization or entity (e.g., a health care company, a research entity, an insurance agency, or the like).

As disclosed herein, the NLP platform 300 can be configured to perform NLP processing techniques to prepare training data of medical information to be used to train AI models in an AI system. Additionally, the NLP platform 300 can maintain a model that the NLP platform 300 may use to prepare the training data. In particular, the NLP platform 300 can be configured to perform NLP processing techniques to de-identify and fictionalize certain medical facts for the training data used in training AI models in an AI system according to the present disclosure. Moreover, the NLP platform 300 can dynamically update the training data as additional inputs, rules, restrictions, etc. are received.

The natural language processing (NLP) platform 300 may include one or more computing devices configured to perform one or more of the functions described herein. For example, the NLP platform 300 may include one or more computers (e.g., servers, server blades, laptop computers, desktop computers, etc.).

The NLP platform 300 can include one or more processors 302, memory 304, and an interface 306. A data bus can interconnect the processor 302, the memory 304, and interface 306. In general, the interface 306 can be a network interface configured to support communication between the NLP platform 300 and one or more networks (not shown).

The memory 304 may include one or more program modules having instructions that when executed by processor 302 cause the NLP platform 300 to perform one or more functions and/or one or more databases that may store and/or otherwise maintain information which the program modules may use and/or the processor 302. In some instances, the one or more program modules and/or databases may be stored by and/or maintained in different memory units of the NLP platform 300 and/or by different computing devices that may form and/or otherwise make up the NLP platform 300. For example, the memory 304 may have, store, and/or include the NLP module 306a, an NLP database 306b, and a machine learning engine 306c.

The NLP platform 300 may have instructions that direct and/or cause the NLP platform 300 to execute advanced natural language processing techniques. For example, the NLP platform 300 can apply NLP techniques to de-identify identifiable facts and to alter (fictionalize) other facts.

The memory 304 may include several components or modules, as illustrated. The NLP database 306b may store information used by the NLP module 306a and/or the NLP platform 300 in de-identifying identifiable facts and altering (fictionalizing) other facts, and/or in performing other functions. The machine learning engine 306c may have instructions that direct and/or cause the NLP platform 300 to perform de-identification steps and fictionalization steps, and to set, define, and/or iteratively refine optimization rules and/or other parameters used by the NLP platform 300 and/or other systems.

The following section generally describes the training an untrained Deep Neural Network (DNN) to generate a trained model using training data according to the present disclosure, such as training an AI model (150) of the discloses system (100) used in formulating interventions based on a patient's context (factual circumstances) as described above. The neural network is trained based on the training data, which comes from the sources discussed above.

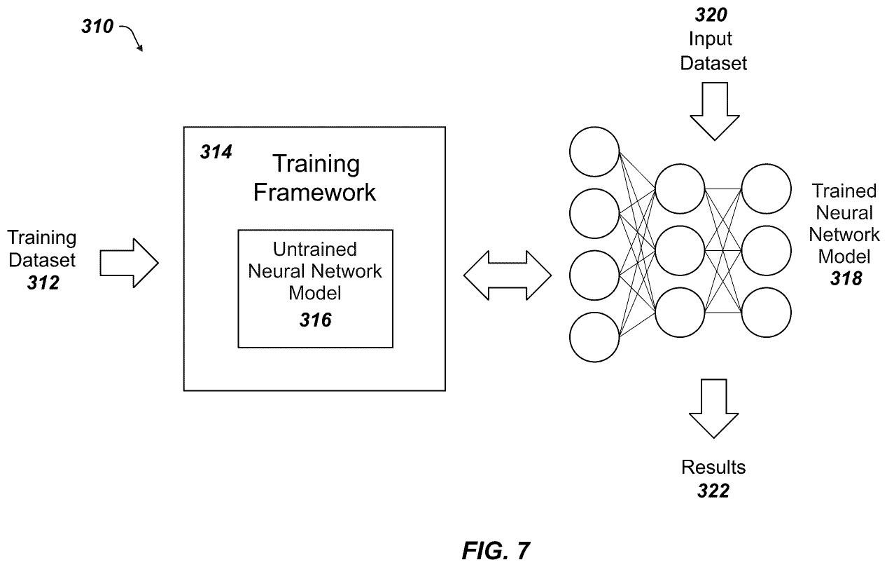

As a further example according to the present disclosure, FIG. 7 illustrates a process for training and deploying a trained neural network model 318 of a deep neural network 310. The following section generally describes the training of an untrained neural network model 316 to generate the trained neural network model 318 using a training dataset 312. The training can be used to train an AI model (150) of the disclosed PoV system (100) used in formulating interventions based on a patient's context (factual circumstances) as described above.

Once the neural network 310 has been structured for a task, the neural network is trained using a dataset of training dataset 312. Initially, the dataset 312 of medical information is processed to remove any personally identifiable facts from the information.

Once the training dataset 312 is available, training of the deep neural network 310 can begin. To begin training the deep neural network 310, initial weights of an untrained model 316 may be chosen randomly or by pre-training using a deep belief network. The training framework 314 then performs a training cycle, which can then be performed in either a supervised or unsupervised manner.

Supervised learning uses the training dataset 312 to teach models to yield the desired output. The training dataset 312 includes inputs and desired outputs, which allow the model to learn over time, or when the training dataset 312 includes input having known output and the output of the neural network is manually graded.

The training framework 314 of the deep neural network 310 processes the inputs and compares the resulting outputs against a set of expected or desired outputs. Errors are then propagated back through the training framework 314. As the training proceeds, the training framework 314 can adjust and change the weights that control the untrained neural network model 316. The training framework 314 can provide tools to monitor how well the untrained model 316 is converging towards a model suitable for generating correct answers based on known input data. The training process repeatedly occurs as the network weights are adjusted to refine the output generated by the neural network. The training process can continue until the neural network reaches a statistically desired accuracy associated with a trained neural network model 318. Ultimately, the trained model 318 can then be deployed in the disclosed PoV system (100) so the trained model 318 presented with an input dataset 320 can implement the machine learning operations and output a result 322 for use according to the purposes of the disclosed PoV system (100).

Supervised learning is typically separated into two types of problems—classification and regression. Classification uses an algorithm to assign test data accurately into specific categories. Regression is used to understand the relationship between dependent and independent variables. Numerous different algorithms and computation techniques can be used in supervised machine learning, including but not limited to, neural networks, naïve bayes, linear regression, logistic regression, support vector machines (SVM), k-nearest neighbor, and random forest.

Unsupervised learning is a learning method in which the network uses algorithms to analyze and cluster unlabeled data. These algorithms discover hidden patterns or data groupings. Therefore, the training dataset 312 includes input data without any associated output data. The untrained neural network model 316 can learn groupings within the unlabeled input and determine how individual inputs relate to the overall dataset.

Unsupervised training can be used for three main tasks—clustering, association, and dimensionality. Clustering is a data mining technique that groups unlabeled data based on similarities and differences. This technique is often used to process raw, unclassified data objects into groups represented by structures or patterns in the information. Association is a rule-based method for finding relationships between variables in a given dataset. This method is often used for market basket analysis. Dimensionality reduction is used when a given dataset's number of features (dimensions) is too high. This technique is commonly used in the preprocessing of data.

Variations of supervised and unsupervised training may also be employed. Semi-supervised learning is a technique in which the training dataset 312 includes a mix of labeled and unlabeled data of the same distribution. Incremental learning is a variant of supervised learning in which input data is continuously used to train the model further. Incremental learning enables the trained neural network model 318 to adapt to the input dataset 320 without forgetting the knowledge instilled within the network during initial training.

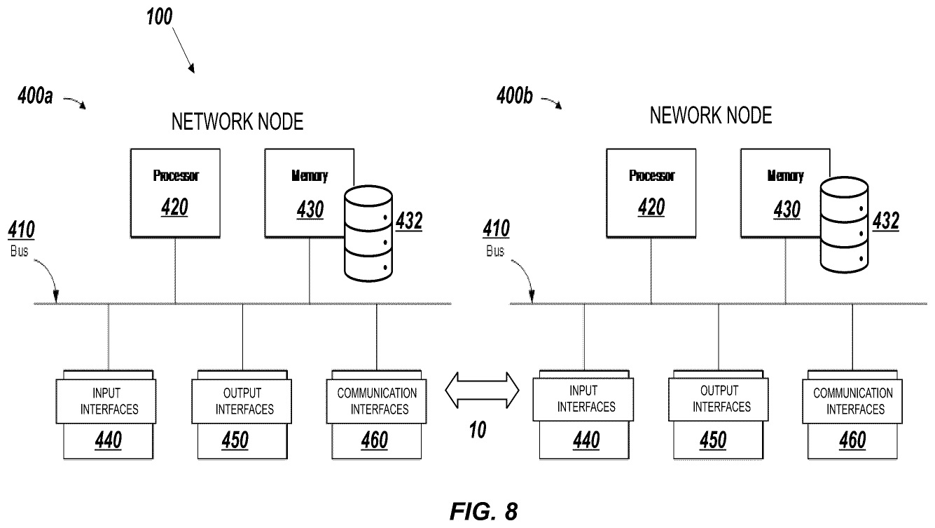

FIG. 8 schematically illustrates components of network devices or nodes 400a-b of the disclosed system 100 in the network system 10 of FIGS. 1-2. As shown in FIG. 8, each network node 400a-b may include a bus 410, a processor 420, a memory 430, and a communication interface 440. Other components, such as an input and output interface 440, 450, may be available.

The bus 410 includes one or more components that enable wired and/or wireless communication among the components of the network node 400a-b. The bus 410 may couple together two or more components of FIG. 8, such as via operative coupling, communicative coupling, electronic coupling, and/or electric coupling. The processor 420 includes a central processing unit, a microprocessor, a controller, a microcontroller, a digital signal processor, a field-programmable gate array, an application-specific integrated circuit, and/or another type of processing component. The processor 420 is implemented in hardware, firmware, or a combination of hardware and software. In some implementations, the processor 420 includes one or more processors capable of being programmed to perform one or more operations or processes described elsewhere herein.

The memory 430 includes volatile and/or nonvolatile memory. For example, the memory 430 may include random access memory (RAM), read only memory (ROM), a hard disk drive, and/or another type of memory (e.g., a flash memory, a magnetic memory, and/or an optical memory). The memory 430 may include internal memory (e.g., RAM, ROM, or a hard disk drive) and/or removable memory (e.g., removable via a universal serial bus connection). The memory 430 may be a non-transitory computer-readable medium. The memory 430 stores information, instructions, and/or software (e.g., one or more software applications) related to the operation of the network node 400a-b. In some implementations, the memory 430 includes one or more memories that are coupled to one or more processors (e.g., the processor 420), such as via the bus 410.

For at least one network node 400a, the input/output interfaces 440, 450 enables the network node 400a to receive input, such as user input and/or sensed input. For example, the input/output interfaces 440, 450 may include a touch screen, a keyboard, a keypad, a mouse, a button, a microphone, a switch, a sensor, etc. The input/output interfaces 440, 450 also enables the network node 400a to provide output, such as via a display, a speaker, etc.

The communication interface 440 enables the network node 400a-b to communicate with other devices via a wired connection and/or a wireless connection. For example, the communication interface 440 may include a receiver, a transmitter, a transceiver, a modem, a network interface card, and/or an antenna.

The network node 400a-b may perform one or more operations or processes described herein. For example, a non-transitory computer-readable medium (e.g., the memory 430) may store a set of instructions (e.g., one or more instructions or code) for execution by the processor 420. The processor 420 may execute the set of instructions to perform one or more operations or processes described herein. In some implementations, execution of the set of instructions, by one or more processors 420, causes the one or more processors 420 and/or the network node 400a-b to perform one or more operations or processes described herein. In some implementations, hardwired circuitry may be used instead of or in combination with the instructions to perform one or more operations or processes described herein. Additionally, or alternatively, the processor 420 may be configured to perform one or more operations or processes described herein. Thus, implementations described herein are not limited to any specific combination of hardware circuitry and software.

As disclosed herein, the PoV system (e.g., distributed network system) 100 operates within one or more networks of a decentralized health information network system 10, connecting with numerous network nodes, which are associated with patients, providers, and payers, to facilitate PoV transaction fulfillment based on a consensus contract. The several components for the disclosed system 100 can be incorporated into one or more of the network nodes 400a-b, can be shared between one or more of the network nodes 400a-b, and/or can be distributed among several of the network nodes 400a-b. (Although two network node 400a-b are shown, it will be appreciated that the network can include any plurality of such devices.) Therefore, the disclosed system 100 generally includes one or more databases 432 (e.g., in memory 430), one or more interfaces (e.g., 440, 450, 460), and one or more processors (e.g., processors 420) incorporated into one or more of the network node 400a-b, shared between several of the network node 400a-b, and/or distributed among several of the network node 400a-b.

The one or more databases 432 in memory 430 store intervention rules, valuation rules, and communication rules, all governed by the consensus contract (50; FIG. 2). These rules dictate interventions in response to medical-related events, attribute valuations to network nodes (11), and define communication protocols with each network node (11). The one or more interfaces 440, 450, 460 are designed to facilitate communication between network nodes (11) across the networks (12).

The one or more processors 420 are in operational communication with the databases 432 and the interfaces 440, 450, 460. These processors 420 are configured to build a distributed ledger (110) for a given patient (20). To do this, the processors 420 receive medical-related events for the given patient (20), add these events to the ledger 110 based on communication rules; select interventions based on intervention rules; communicate these interventions to relevant network nodes (11); record the interventions in response to the communication; and evaluate the interventions' value for the involved members (e.g., patient, provider, etc.) according to the valuation rules. Subsequently, the assessed valuations are added to the distributed ledger (110).

Furthermore, the processors 420 are configured to distribute the distributed ledger (110) within the health information network system 10. Additionally, the processors 420 are configured to arrange settlements (44) or rewards (46) for the assessed valuations among the relevant members (e.g., patient, provider, etc.) in accordance with the consensus contract (50). Overall, the disclosed system 100 actively performs these functions to ensure seamless operation and adherence to the established consensus contract (50).

The number and arrangement of components shown in FIG. 8 are provided as an example. The network node 400a-b may include additional components, fewer components, different components, or differently arranged components than those shown in FIGS. 8. Additionally, or alternatively, a set of components (e.g., one or more components) of the network node 400a-b may perform one or more functions described as being performed by another set of components of the network node 400a-b.

The techniques of the present disclosure can be implemented in digital electronic circuitry, or in computer hardware, firmware, software, or in combinations of these. Apparatus for practicing the disclosed techniques can be implemented in a computer program product tangibly embodied in a machine-readable storage device for execution by a programmable processor; and method steps of the disclosed techniques can be performed by a programmable processor executing a program of instructions to perform functions of the disclosed techniques by operating on input data and generating output. The disclosed techniques can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random-access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

The foregoing description of preferred and other embodiments is not intended to limit or restrict the scope or applicability of the inventive concepts conceived of by the Applicants. It will be appreciated with the benefit of the present disclosure that features described above in accordance with any embodiment or aspect of the disclosed subject matter can be utilized, either alone or in combination, with any other described feature, in any other embodiment or aspect of the disclosed subject matter.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. A method, used in a distributed network system between a plurality of network nodes in a healthcare environment, to fulfill proof of value transactions according to a consensus contract, the network nodes being at least associated with one or more members in the healthcare environment that include patients, providers, and payers, the method comprising
storing, in one or more databases of the distributed network system, (i) a plurality of intervention rules governed by the consensus contract, the intervention rules defining a plurality of interventions in response to events, (ii) a plurality of context rules defining context indicators for factual circumstances associated with the patients, and (iii) a plurality of communication rules governed by the consensus contract, the communication rules defining a plurality of communications with each of the network nodes for the interventions;
building, with one or more processors of the distributed network system, a distributed ledger for a given patient by:
receiving a profile having one or more of the factual circumstances associated with the given patient;
attributing one or more context indicators for the one or more factual circumstances in the profile based on the context rules;
adding the one or more context indicators to the distributed ledger;
receiving a given event for the given patient;
adding the given event to the distributed ledger based on the communication rules;
selecting a first intervention based on the one or more context indicators;
communicating the first intervention, as a first communicated intervention, to a given one or more of the network nodes based on the communication rules;
receiving a first record of the first communicated intervention, as a first recorded intervention, in response to the communication; and
adding the first recorded intervention to the distributed ledger in response to the first record; and
distributing the distributed ledger in the distributed network system.

2. The method of claim 1, wherein the intervention rules defining the interventions in response to the events define how each of the events are detected and how the interventions are subsequently selected.

3. The method of claim 1, wherein the communication rules defining the communications with each of the network nodes for the interventions define how the distributed network system interfaces with each of the network nodes in sending and receiving the communications.

4. The method of claim 1, wherein receiving the given event for the given patient comprises receiving an observation of the given patient, the given patient having a stated medical condition, thereby adding the observation for the stated medical condition to the distributed ledger.

5. The method of claim 1, wherein:
selecting comprises selecting a directed action for a given provider, as the first selected intervention, based on the one or more context indicators;
communicating comprises communicating the directed action, as the first communicated intervention, to the given provider based on the communication rules;
receiving comprise receiving an indication of completion of the directed action, as the first record, in response to the communication; and
adding comprises adding the indication of completion of the directed action, as the first recorded intervention, to the distributed ledger in response to the indication.

6. The method of claim 1, wherein building the distributed ledger for the given patient further comprises:
selecting a second intervention, as a second selected intervention, in response to the given event based on the intervention rules;
communicating the second selected intervention, as a second communicated intervention, to a given one of the network nodes based on the communication rules;
receiving a second record of the second communicated intervention, as a second recorded intervention, in response to the communication; and
adding the second recorded intervention to the distributed ledger in response to the second record.

7. The method of claim 6, wherein:
selecting comprises selecting a directed action for the given patient, as the second selected intervention, based on the intervention rules;
communicating comprises communicating the directed action, as the second communicated intervention, to the given patient based on the communication rules;
receiving comprise receiving an indication of completion of the directed action, as the second record, in response to the communication; and
adding comprises adding the indication of completion of the directed action, as the second recorded intervention, to the distributed ledger in response to the indication.

8. The method of claim 1, further comprising storing, in the one or more databases, (iv) a plurality of valuation rules governed by the consensus contract, the valuation rules attributing a valuation for each of the members for the interventions.

9. The method of claim 8,
wherein building the distributed ledger for the given patient comprises:
assessing one or more first valuations, as one or more first assessed valuations, of the first recorded intervention for a given one or more of the members based on the valuation rules; and
adding the one or more first assessed valuations to the distributed ledger; and
wherein the method further comprises:
configuring a settlement of the one or more first assessed valuations for the given one or more of the members according to the consensus contract by distributing the distributed ledger in the distributed network system.

10. The method of claim 9, wherein configuring the settlement by distributing the distributed ledger comprises:
adding data including the given event, the recorded intervention, and the one or more first assessed valuations in blocks to the distributed ledger and including, in each block, a unique cryptographic hash of a previous block in the distributed ledger; and
automatically fulfilling terms of the consensus contract in response to predefined conditions being met by coding terms of the consensus contract into code used in the network.

11. The method of claim 8,
wherein building the distributed ledger for the given patient further comprises:
selecting a second intervention, as a second selected intervention, in response to the given event based on the intervention rules;
communicating the second selected intervention, as a second communicated intervention, to a given one of the network nodes based on the communication rules;
receiving a second record of the second communicated intervention, as a second recorded intervention, in response to the communication;
adding the second recorded intervention to the distributed ledger in response to the second record;
assessing one or more second valuations, as one or more second assessed valuations, of the second recorded intervention for a given one or more of the members based on the valuation rules; and
adding the one or more second assessed valuations to the distributed ledger; and
wherein the method further comprises:
configuring a settlement of the one or more second assessed valuations for the given one or more of the members according to the consensus contract by distributing the distributed ledger in the distributed network system.

12. The method of claim 8, wherein the valuation rules attributing the valuation for each of the members for the interventions define how the first recorded interventions are detected and how the first recorded interventions are subsequently valued.

13. The method of claim 1, wherein attributing the one or more context indicators for the one or more factual circumstances in the profile based on the context rules comprises analyzing one or more of demographic information, patience resilience, and social determinants of the given patient contained in the profile.

14. The method of claim 1, wherein the context indicators for the factual circumstances associated with the patients are selected from the group consisting of:
demographic information, patient resilience, type of medical condition diagnosed, time since diagnosis of medical condition, severity of medical condition, progression of medical condition, housing, food, nutrition, transportation, social mobility, economic mobility, education, environmental condition, extent of caregiving network, Social Determinants of Health (SDoH) information, and Health-Related Social Need (HSRN) information.

15. The method of claim 1, wherein to select the first intervention based on the one or more context indicators, the method comprises using an artificial intelligence (AI) model trained by training data including the factual circumstances associated with a plurality of the patients.

16. The method of claim 15, wherein to use the AI model, the method comprises:
training the AI model to detect a plurality of subtle scenarios from the context indicators and to indicate alternative courses of actions for the subtle scenarios; and
informing the intervention rules based on at least one of fixed thresholds and the AI model to formulate the interventions for each of the members.

17. The method of claim 1, wherein the providers include one or more of a hospital, a doctor, a medical supplier, etc.; wherein the payers include one or more of health management company, a medical insurer, an insurance company, a health plan administrator, etc.; wherein the events include one or more of: a medical-related event, a therapeutic-related event, a diagnosis, a medical observations, a consumption of a medical supply, hospitalization of a care-giver, and a change to a patient's circumstances like financial, housing, or food security; and wherein the interventions include one or more of a therapeutic protocol, a directed action,, an adjustment to care plan, an educational material to raise condition literacy, an assistance to raise awareness about social program (like ride assistance or meal delivery), a delivery of a medical supply, and a reward for achieving therapeutic goals (like points programs, or reduced insurance cost).

18. A programmable storage device having program instructions stored thereon for causing one or more programmable network devices to perform a method of claim 1, used in a distributed network system between a plurality of network nodes, to fulfill transactions according to a consensus contract.

19. A distributed network system used in one or more networks between a plurality of network nodes to fulfill proof of value transactions according to a consensus contract, the network nodes being at least associated with members in a healthcare environment that includes patients, providers, and payers, the distributed network system comprising:
one or more databases storing (i) a plurality of intervention rules governed by the consensus contract, the intervention rules defining a plurality of interventions in response to events, (ii) a plurality of context rules defining context indicators for factual circumstances associated with the patients, and (iii) a plurality of communication rules governed by the consensus contract, the communication rules defining a plurality of communications with each of the network nodes for the interventions;
one or more interfaces configured to communicate the network nodes with one another via the one or more networks; and
one or more processors in operable communication with the one or more databases and the one or more interfaces,
wherein, to build a distributed ledger for a given patient, the one or more processors are configured to:
receive a given profile having one or more of the factual circumstances associated with the given patient;
attribute one or more context indicators for the one or more factual circumstances in the given profile based on the context rules;
add the one or more context indicators to the distributed ledger;
receive a given event for the given patient;
add the given event to the distributed ledger based on the communication rules;
select a first intervention based on the one or more context indicators;
communicate the first intervention, as a first communicated intervention, to a given one or more of the network nodes based on the communication rules;
receive a first record of the first communicated intervention, as a first recorded intervention, in response to the communication; and
add the first recorded intervention to the distributed ledger in response to the first record,
wherein the one or more processors are configured to distribute the distributed ledger in the distributed network system.
